(19) United States
(12) Patent Application Publication
(10) Pub. No.: US 2009/0312830 A1
(43) Pub. Date: Dec. 17, 2009

(54) Balloon arc profile control

(76) Inventors: Sean McNulty, Cottage Grove, WI (US); David Broman, Rogers, MN (US); Matthew Heidner, Maple Grove, MN (US); Tracee Eidenschink, Wayzata, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 12/214,253

(22) Filed: Jun. 17, 2008

(51) Int. Cl. A61F 2/06

(52) U.S. Cl. 623/1.11; 623/1.17; 623/1.44; 623/1.49

(57) ABSTRACT

The present invention is directed to a delivery system including a stent protector to protect an end of the stent and/or stent body for delivery of the stent to an intended fixation site or treatment site within a body lumen. More specifically, the present invention is directed to balloon catheter which protects the distal end, proximal end and/or body of a stent during delivery to the deployment site and/or shipping of a preloaded system.

### FIELD OF THE INVENTION

The present invention relates generally to a system and method of delivering an endoluminal prosthesis within a body lumen. More particularly, the present invention is directed to device for delivering a stent to a lumen.

### BACKGROUND OF THE INVENTION

Expandable, implantable medical devices such as stents are utilized in a number of medical procedures and situations as are stent delivery assemblies. As such, their structure and function are well known. A stent is a generally cylindrical prosthesis introduced via a catheter into a lumen of a body vessel in a configuration having a generally reduced diameter and then expanded to the diameter of the vessel. The stent may be self-expanding, for example, the stent may comprise a super elastic and/or linear elastic material such as nickel-titanium alloy (Nitinol), or it may be expandable by means of an inflatable portion of the catheter, such as a balloon. In its expanded configuration, the stent supports and reinforces the vessel walls while maintaining the vessel in an open, unobstructed condition.

Balloon expandable and balloon assisted expandable stents are expanded via outward radial pressure such as that provided by a balloon disposed underneath the stent during inflation of the balloon.

In advancing a balloon expandable stent through a body vessel to the deployment site, there are a number of important considerations, such as delivery device size, stent parameters, condition of deployment site. Medical device delivery balloons may have a variety of shapes, sizes, inflation characteristics and a variety of other performance attributes. The catheter helps to atraumatically advance the system and protects the stent. These stents can be delivered into the lumen using a system which includes a catheter, with the stent supported near its distal end, and a sheath, positioned coaxially about the catheter and over the stent, to prevent abrasion between the stent and body wall as the catheter is directed through torturous body pathways.

Once the stent is located at the constricted portion of the lumen, the sheath is removed to expose the stent, which is expanded so it contacts the lumen wall. The catheter is subsequently removed from the body by pulling it in the proximal direction, through the larger lumen diameter created by the expanded prosthesis, which is left in the body.

The stent ends are often damaged during delivery through the catheter, or shipping of a pre-loaded delivery device. Thus it is desirable to protect the distal and proximal ends of the stent to prevent distortion of the stent and to prevent abrasion and/or to reduce potential trauma to the vessel walls.

### SUMMARY OF THE INVENTION

The present invention provides a stent delivery system including a balloon catheter, a stent protector, and a stent. The balloon catheter has a balloon and outer sheath and the balloon has a distal end and a proximal end. The stent protector extends partially about the distal end of the balloon. The stent extends about a portion of the balloon at the proximal end of the balloon. The stent protector is spaced apart from the stent. The balloon arches radially between the stent protector and the stent.

Another embodiment of the present invention includes a stent edge protector including a stent having a first end, second end and a body therebetween; and a deflated catheter balloon having a distal end section, middle arching section, and a proximal end section. The stent is mounted over the proximal section. The distal end section has a reduced diameter. The middle arching section has an arc profile with a larger diameter then the proximal end section and distal end section. The first end of the stent abuts the middle arching section and the middle arching section provides protection to the first end of the stent.

A further embodiment includes a method of making a stent protector for protecting a stent from damage during shipment and implantation. The method includes the steps of providing a balloon catheter including an outer sheath and inflatable balloon for supporting a stent thereon, the balloon has a distal end and a proximal end; providing a stent; providing a stent protector; mounting a stent on the balloon; placing an outer sheath over the balloon and the stent; pushing the stent protector over the balloon at the distal end, excess balloon material being pushed toward the stent and away from the stent protector. The excess material radially arches outwardly toward the outer sheath between the stent protector and the stent; and abutting the arching excess material with the stent. Further, a heating step may be added to allow the balloon arching portion to expand radially outward toward the outer sheath; and cooling the balloon to permanently set the balloon in a arching profile between the stent and the stent protector, and set the balloon with a reduced diameter within the stent protector. The stent protector may be removed from the balloon after the heat setting and the balloon retains the arching profile and the reduced profile.

### BRIEF DESCRIPTION OF THE DRAWINGS

FIG. 1 is a perspective side view showing a portion of a delivery device of the present invention.

FIG. 2 is a cross-sectional side view of a stent protector 20 affect on the profile of a balloon cone of the present invention.

FIGS. 3-5 is a perspective side view showing varying distal balloon cone diameters from the present invention.

### DETAILED DESCRIPTION OF THE PREFERRED EMBODIMENTS:

The present invention is directed to a delivery system 10 including a stent protector 20 to protect an end of the stent 18 and/or stent body for delivery of the stent 18 or stent-graft to an intended fixation site or treatment site within a body lumen. More specifically, the present invention is directed to balloon catheter 11 which protects the distal end, proximal end and/or body of a stent 18 during delivery to the deployment site and/or shipping of a preloaded system.

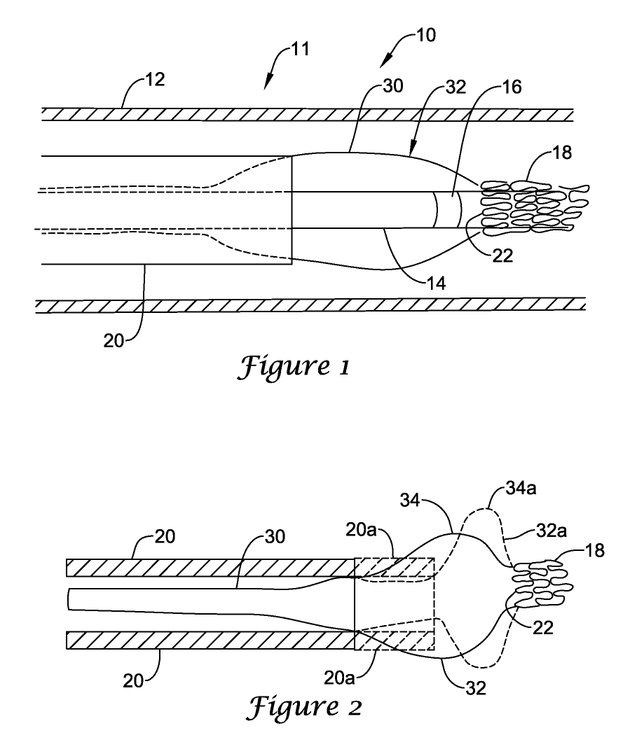

Referring to FIG. 1, a stent delivery system 10 of the present invention includes balloon catheter 11 and a stent protector 20. The balloon catheter 11 includes an outer sheath 12 and an inflatable balloon 30 for supporting a stent 18. The balloon 30 is fixed to the catheter 11 for remote inflation as is known in the art. The catheter 11 includes an elongate cannula 14 and may include marker bands 16. The balloon 30 extends through a stent protector 20 and a stent 18. The stent protector 20 and the stent 18 are spaced apart from each other leaving a portion of balloon 30 uncovered therebetween. The stent protector 20 is sized tightly around the balloon 30 to manipulate and control the size and profile of the balloon arc 32 adjacent to the stent edge 22. The outer sheath 12 encapsulates the stent 18, balloon 30 and stent protector 20.

FIGS. 1-5 show a stent 18; however, various balloon expandable prostheses may be employed in the present invention. A stent, stent covered graft, or other stent/graft combinations may be employed as known in the art. Among the various stents that may be employed, there are a host of geometries and materials of construction to choose from, as known in the art. Some suitable stent metallic materials include, but are not necessarily limited to, stainless steel, tantalum, tungsten, nickel-titanium alloys such as those possessing shape memory properties commonly referred to as nitinol, nickel-chromium alloys, nickel-chromium-iron alloys, cobalt-chromium-nickel alloys, or other suitable metals, or combinations or alloys thereof. Some suitable stent polymeric materials include, but are not necessarily limited to polyamide, polyether block amide, polyethylene, polyethylene terephthalate, polypropylene, polyvinylchloride, polyurethane, polytetrafluoroethylene, polysulfone, and copolymers, blends, mixtures or combinations thereof.

Balloon 30 may be composed of any thermoplastic polymer, or polymers, suitable for use as a medical balloon. Balloon 30 may be a unitary body of material continuity, or may be a combination of various materials with various characteristics to provide additional elasticity in a middle arching section. Such thermoplastic polymers include, but are not limited to: polyethylene teraphtholate (PET), polybutylene teraphtholate (PBT), PEBAX™, Nylon™, polyurethane, polyester-polyether block copolymer such as ARNITEL™, polyolefin and polyolefin compounds. In addition, the present balloon may be manufactured by any means appropriate for manufacture a medical balloon as known in the art. The balloon may have a variety of geometries as known in the art. The balloon may be made from typical angioplasty balloon materials including polymers such as polyethylene terephthalate (PET), polyetherimide (PET), polyethylene (PE), etc. Some other examples of suitable polymers, including lubricious polymers, may include polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, and a polyether-ester elastomer such as ARNITEL( available from DSM Engineering Plastics).

Additional examples of suitable polymers include polyester (for example, a polyester elastomer such as HYTREL® available from DuPont), polyamide (for example, DURETHAN® available from Bayer or CRISTAMIDO® available from Elf Atochem), elastomeric polyamides, block polyamide/ethers, nylons such as polyether block amide (PEBA, for example, available under the trade name PEBAX®), silicones, Marlex high-density polyethylene, Marlex low-density polyethylene, linear low density polyethylene (for example, REXELL®), polyetheretherketone (PEEK), polyimide (PI), polyphenylene sulfide (PPS), polyphenylene oxide (PPO), polysulfone, nylon, perfluoro(propyl vinyl ether) (PFA), other suitable materials, or mixtures, combinations, copolymers thereof, polymer/metal composites, and the like.

FIGS. 1-5 show stent protector 20 as a inner tube with a diameter that is smaller than the outer sheath 12 but large enough to allow a portion of the balloon 30 to pass therethrough. The diameter of the stent protector 20 varies depending on the size of the stent 18, balloon 30 and the catheter 11.

Alternatively, the stent protector 20 may be a tube, clamp, washer, necking die, ring, clamshell or the like. The stent protector 20 is used to form a balloon arc profile 32. The stent protector 20 may be made from a variety of materials which will not damage, deteriorate, react or contaminate the balloon 30, stent 18 or catheter 11. Some materials which may be used in the stent protector 20 are similar to the materials of construction of an outer sheath, which include, but are not limited to polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers such as HYTREL® available from DuPont), polyamide (for example, DURETHAN® available from Bayer or CRISTAMID® available from Elf Atochem), elastomeric polyamides999, block polyamide/ethers, polyether block amide (PEBA, for example available under the trade name PEBAX®), ethylene vinyl acetate copolymers (EVA), silicones, polyethylene (PE), Marlex high-density polyethylene, Marlex, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example KEVLAR®), polysulfone, nylon, nylon-12 (such as GRILAMID® available from EMS American Grilon), perfluro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6% LCP.

The stent protector 20 includes a lubricous and smooth inner surface to allow the protector to move over the balloon 30 without damaging it. The outer surface is also preferably smooth to prevent damage to the outer sheath 12. The stent protector 20 moves within the outer sheath 12 allowing for a space between the stent protector 20 and the outer sheath 12. The stent protector 20 preferably does not contact the outer sheath 12 inner surface.

The stent protector 20 has a tightly tolerance diameter and is positioned over the balloon 30 creating a snow plow affect defined as pushing the balloon 30 material away from the stent protector 20 and toward the stent edge 22. FIG. 2 shows the snow plow affect, the material of the balloon 30 expands radially outward creating the arc profile 32, 32a because the excess balloon 30 material is prevent from being displacing within the stent 18. The protection of the edge of the stent 18 improves the closer the peak 34, 34a of the balloon arc profile 32, 32a is to the stent edge 22. The optimal distance between the peak 34 of the balloon arc profile 32 and the stent edge 22 varies depending on the sizes of the delivery device components. The peak 34 includes a diameter which is larger than the diameter of the stent protector 20, but smaller than the diameter of the outer sheath. The profile, shape and size of the arc 32 can be controlled by changing the amount of balloon 30 material that is inside of the small diameter tube of the stent protector 20, 20a, diameter of the stent protector 20, and/or the distance from the end of the stent protector 20 to the stent end or stent edge 22, as shown in FIGS. 2-4.

Additionally, the stent protector 20 does not contact the stent edge 22 because it will damage the stent edge 22. There must be a distance between the stent edge 22 and stent protector 20 to allow for the snow plow affect. The arc profile 32 may be heat set to fix the arc 32 in the desired position and profile. The stent protector 20 holds the balloon 30 in a compressed state during sterilization. The portion of the balloon 30 that is not restricted by the stent protector 20 relaxes and expands during sterilization. The relaxing of the balloon 30 creates an radially outward expansion to form an arc profile 32 with a peak 34 between the stent protector 20 and the stent edge 22. The portion of the balloon 30 within the stent protector 20 remains in a compressed state or tightly wrapped. The stent protector 20 may be left on during shipment of a loaded device 10. The stent protector 20 is removed before use of the delivery system 10 to implant the stent 18. Further, the stent protector 20 may be removed after heat setting or sterilization. The stent edge 22 is protected by the excess balloon material around the stent edge 22 from the arc profile 32. The stent 18 body is prevented from contacting the outer sheath 12 because of the larger diameter of the arc profile 32 especially about the peak 34.

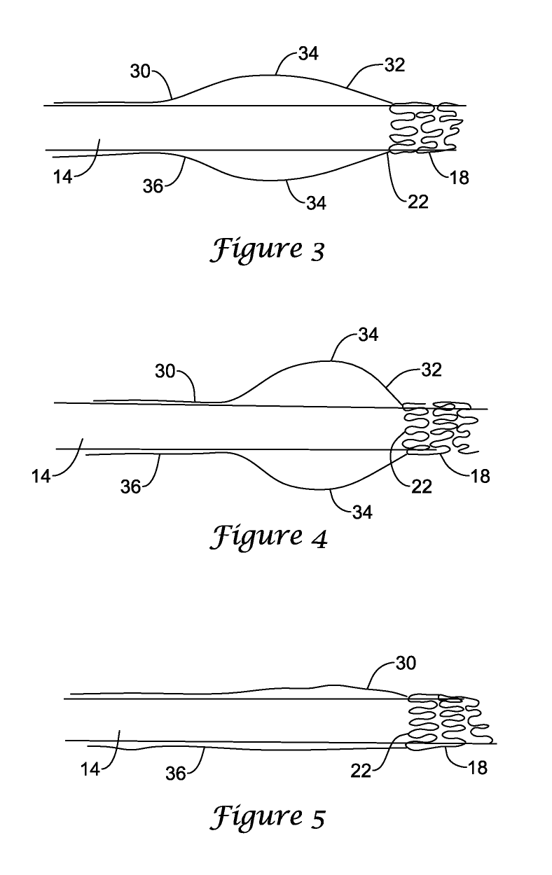

FIGS. 3-5 shows a variety of arc profiles 32 of a balloon 30 having a cone end 36 geometry from the snow plow affect after sterilization and removal of the stent protector 20. FIG. 3 shows the affect of the stent protector 20 which the stent protector 20 comes just up to the distal cone end 36 of a balloon 30. The arc 32 is elongated and the peak 34 is a distance from the stent edge. A minimal amount of balloon 30 material was displaced to for the arc 32. FIG. 4 shows the affect of the stent protector 20 which comes roughly halfway up the distal cone end 36 and places the peak 34 of the balloon arc profile 32 closer to the stent edge 22. Notice the peak 34 is further from the cannula creating a shorter and higher arc closer to the stent edge 22. The peak 34 moved proximally in comparison to FIG. 1. FIG. 5 shows the affect of a stent protector 20 which was placed up to almost the distal edge of the stent 18 preventing balloon 30 from creating an arc 32. The profile of FIG. 5 is much flatter, with no peaking and arching when compared to FIGS. 3 and 4. The stent edge 22 is left exposed to the elements in FIG. 5, unlike FIGS. 3 and 4.

The balloon arc profile 32 protects the body of the stent 18 from contacting the surface of the outer sheath 12 which may cause damage to the stent 18 or coating thereon. The balloon arc 32 has a larger diameter then the stent 18. The balloon arc 32 creates a gap between the outer sheath 12 and the stent body. The balloon arc 32 will contact the outer sheath 12 and limit the movement of the stent 18 within the outer sheath 12 providing for stent 18 body and stent edge 22 protection.

Having described the preferred embodiments herein, it should now be appreciated that variations may be made thereto without departing from the contemplated scope of the invention. Accordingly, the preferred embodiments described herein are deemed illustrative rather than limiting, the true scope of the invention being set forth in the claims appended hereto.

1. A stent delivery system comprising:
a balloon catheter including a balloon, said balloon having a distal portion, a middle arching portion, and a proximal portion;
a stent protector disposed partially about said distal portion of said balloon; and
a stent disposed about the proximal portion of said balloon, said stent having a first end, a second end, and a body extending therebetween;
wherein said stent protector is spaced apart from said stent, said middle arching portion arching radially outward between said stent protector and said stent.

2. The stent delivery system of claim 1 wherein said stent protector is sized tightly around said balloon.

3. The stent delivery system of claim 1 wherein said stent protector is a washer.

4. The stent delivery system of claim 1 further comprising an outer sheath disposed about the balloon and stent, wherein said stent protector is a tube having an outer diameter smaller than an inner diameter of said outer sheath.

5. The stent delivery system of claim 1 wherein said stent protector is a clamp.

6. The stent delivery system of claim 1 wherein said stent protector comprises a polymer.

7. The stent delivery system of claim 1 wherein said stent protector is metallic.

8. The stent delivery system of claim 1 wherein said stent protector is made from a material selected from the group consisting of polytetrafluoroethylene (PTFE), ethylene tetrafluoroethylene (ETFE), fluorinated ethylene propylene (FEP), polyoxymethylene (POM), polyether block ester, polyurethane, polypropylene (PP), polyvinylchloride (PVC), polyether-ester, ether or ester based copolymers, polyamide, elastomeric polyamides, block polyamide/ethers, polyether block amide (PEBA), ethylene vinyl acetate copolymers (EVA), silicones, polyethylene (PE), high-density polyethylene, low-density polyethylene, linear low density polyethylene, polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide, polysulfone, nylon, nylon-12, perfluro(propyl vinyl ether) (PFA), ethylene vinyl alcohol, polyolefin, polystyrene, epoxy, polyvinylidene chloride (PVdC), polycarbonates, ionomers, liquid crystal polymer (LCP), and combinations thereof.

9-10. (canceled)

11. The stent delivery system of claim 1 wherein
said middle arching portion includes an arc profile defining a peak with a larger outer diameter then said proximal portion, said distal portion, and said stent protector, wherein said peak is located between said stent and said stent protector;
said first end of said stent abutting said middle arching portion and said middle arching portion providing protection to said first end of said stent.

12-14. (canceled)

15. The stent delivery system of claim 1 wherein said distal portion, said middle arching portion and said proximal portion is a unitary body of material continuity.

16. The stent delivery system of claim 1 wherein said middle arching portion is formed of a different material than said distal portion and proximal portion to provide more elasticity in said middle arching portion.

17. (canceled)

18. A method of making a stent delivery system, the method comprising the steps of:
providing a balloon catheter including an outer sheath and an inflatable balloon for supporting a stent thereon, said balloon having a distal end and a proximal end;
providing a stent;
providing a stent protector;
mounting the stent on said balloon;
advancing said stent protector over said balloon at said distal end such that excess balloon material is pushed away from said stent protector as said stent protector is advanced thereby defining an excess material section, said excess material section radially arching outwardly between said stent protector and said stent; and
abutting said excess material section with said stent.

19. The method of claim 18 further including the steps of heating said balloon to allow said excess material section to further expand radially outward; and cooling said balloon to permanently set said excess material section in an arching profile between said stent and said stent protector, and set said balloon with a reduced diameter profile within said stent protector.

20. The method of claim 19 further including the step of removing the stent protector from the balloon, said balloon retaining said arching profile and said reduced profile.

21. The stent delivery system of claim 4, wherein said stent protector is configured to move within said outer sheath and does not contact an inner surface of said outer sheath.

22. The stent delivery system of claim 1, wherein said stent protector has an inner surface configured to allow said stent protector to move over said distal portion of said balloon without damaging said balloon.

23. The method of claim 18 further including the step of placing an outer sheath over said balloon and said stent.

24. The method of claim 18, wherein said excess material section has an outer diameter that is greater than an outer diameter of said stent, an outer diameter of said balloon proximal end, and an outer diameter of said balloon distal end.

25. The method of claim 18, wherein during said advancing step, said excess balloon material is prevented from being displaced within said stent.

26. A method of making a stent delivery system, the method comprising the steps of:
providing a balloon catheter including an inflatable balloon for supporting a stent thereon, said balloon having a distal end and a proximal end;
providing a stent;
providing a stent protector;
mounting the stent on said balloon;
placing an outer sheath over the balloon and the stent;
advancing the stent protector proximally over the distal end of the balloon and within the outer sheath such that excess balloon material is pushed away from the stent protector as the stent protector is advanced thereby defining an excess material section, the excess material section radially arching outwardly toward the outer sheath between the stent protector and the stent;
abutting the excess material section with the stent;
heating the balloon to allow the excess material section to further expand radially outward toward the outer sheath;
cooling the balloon to permanently set the excess material section in an arching profile between the stent and the stent protector, and set the balloon with a reduced diameter profile within the stent protector; and
removing the stent protector from the balloon, the balloon retaining the arching profile and the reduced profile, the arching profile having a greater outer diameter than the reduced profile and the stent;
wherein after removing the stent protector from the balloon, the greater outer diameter of the arching profile provides a gap between an inner surface of the outer sheath and an outer surface of the stent, thereby preventing contact between the stent and the outer sheath.
